(19) United States
(12) Patent Application Publication
(10) Pub. No.: US 2023/0145699 A1
(43) Pub. Date: May 11, 2023

(54) RAPID POINT OF CARE ASSAY FOR THE DETECTION OF THE ASYMPTOMATIC CARRIER STATE OF COVID-19

(71) Applicant: Lawrence LOOMIS, Columbia, MD (US)

(72) Inventor: Lawrence LOOMIS, Columbia, MD (US)

(21) Appl. No.: 17/916,237

(22) PCT Filed: Apr. 5, 2021
(86) PCT No.: PCT/US21/25767

§ 371 Date: Sep. 30, 2022

Related U.S. Application Data
(60) Provisional application No. 63/005,092, filed on Apr. 3, 2020.

(51) Int. Cl. G01N 33/569; G01N 33/543

(52) U.S. Cl. CPC G01N 33/56983; G01N 33/54388; G01N 2469/20; G01N 2333/165

(57) ABSTRACT

The present invention relates to lateral flow assay devices adapted to detect IgA specific for SARS-CoV-2 in biological samples from subjects suspected to have COVID-19, and methods of using the lateral flow assay devices.

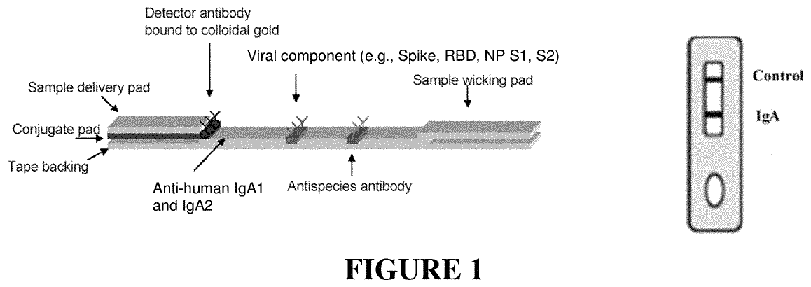

### CROSS REFERENCE TO PRIOR APPLICATIONS

The present application is a U.S. National Phase Application under 35 U.S.C. § 371 of International Patent Application No. PCT/US21/25767 filed Apr. 5, 2021, which claims benefit of priority to U.S. Provisional Patent Application No. 63/005,092 filed Apr. 3, 2020. The entire contents of which are hereby incorporated by reference.

### FIELD OF THE INVENTION

Lateral Flow tests have been developed for the presence of mucosal IgA1 and IgA2 in nasal swabs obtained from the nasopharynx and from saliva which are directed against various SARS-CoV-2 components (Spike protein, receptor binding domain (RBD), nucleocapsid, S1 and S2). In addition, viral peptides that have been identified by mapping mucosal IgA responses to the identified COVID-19 carriers are used to uncover the IgA targeted response to these peptides. The detection of mucosal IgA is the individual's earliest response due to viral exposure and its presence can identify the asymptomatic carrier stage. In addition, monitoring over time the level of mucosal IgA in saliva samples in post vaccinated recipients may determine the probability of post vaccinated recipients to become asymptomatic carriers of the virus.

### BACKGROUND

Coronaviruses are a large family of viruses, including some that cause “the common cold” in healthy humans. They account for up to 30 percent of upper respiratory tract infections in adults. An individual who presents flu like symptoms is understandably concerned he may have Covid-19. More concerning is that many individuals infected with Covid-19 may be asymptomatic and yet are infectious. This is a significant issue around the world, including how to efficiently screen asymptomatic individuals and how to characterize them to limit the spread of the virus.

SARS-CoV-2 is the seventh coronavirus known to infect humans; SARS-CoV, MERSCoV and SARS-CoV-2 can cause severe disease, whereas HKU1, NL63, OC43 and 229E are associated with mild symptoms. The sequences of the SARS-Cov-2 are 96% identical to SARS-CoV and are similarly identical at the whole-genome level to a bat coronavirus. This may cause confusion or misidentification of Covid-19 patients in certain immunological assays and be a potential issue in the future with the advent of other coronaviruses emerging from mixing of species.

The portal of entry of respiratory viruses is via the mucous membrane of the nasopharynx. IgA antibody located on the mucous membrane provides an immune barrier to prevent a virus from penetrating the mucosal lining. IgA is produced locally by large numbers of IgA plasma cells distributed in all subepithelial spaces. Liew et al. (European J. of Immunology 1984) reported that the cross protection of mice infected with influenza A virus by the respiratory route is correlated with local IgA antibody rather than serum antibody or cytotoxic T cell reactivity. The detection of SARS-CoV-2 has been done by molecular assays which have had varying degrees of success depending upon the site that the clinical sample was taken from, or even the producer of the assay (sensitivities of 40%-80% have been reported). Interestingly the percentage for detection of the virus has reportedly been higher in saliva samples compared to samples obtained from the nasopharynx or other sites. It has also been reported that asymptomatic patients often have a relatively high concentration of virus in the nasopharynx, even compared to some symptomatic individuals. It is not understood why this occurs. Serological assays are currently under development for the detection of IgM and IgG in the serum of recovering patients. These tests, however, are based upon the patient's immune response against SARS-CoV-2 antigens, some of which may be shared by other coronaviruses. Furthermore, serum antibodies, especially IgG, can be long lived and therefore while their detection is useful in understanding the mapping of the exposure to the disease, it might have no relationship to the current asymptomatic carrier state.

The SARS-CoV-2 virus enters the cell via ACE2 receptors that line the respiratory system, intestinal tract and all blood vessels. It is the Spike protein and the associated RBD portion that attaches to the ACE2 receptor. It has been found that all recovery patients have antibody directed against the Spike protein as well as the RBD. It has also been found that additional sites on the virus such as the nucleocapsid, S1 and S2 components of the Spike protein, as well as other viral cell membrane components, also have antibody reactive sites.

It has been found that, due to the Spike protein, the SARS-Cov-2 also appears to bind human cells more tightly than the SARS virus, which may help explain why the new coronavirus appears to spread more easily from person to person, mainly by respiratory transmission. Efforts are underway to use the stabilized Spike protein as a probe to isolate naturally produced antibodies from people who have recovered from COVID-19 as the basis of a treatment.

The percent of the population that has IgA deficiency may be as high as 10%. Interestingly it is estimated that about 10% of the population may have serious effects when they become infected with the SARS-CoV-2 virus. Additionally, PCR testing has found the presence of the virus in fecal samples due to the ACE2 receptors lining the intestinal tract. The production of IgA antibodies in the intestine is often expressed in the saliva of infected persons.

The detection of targeted mucosal IgA magnifies the strength of the test's signal (IgA antibody vs COVID-19) and can be more readily detected than the response of PCR antigen testing. This IgA response thus becomes the marker for contact with the virus and signals a possible time when the exposed individual is an asymptomatic carrier.

A test for the presence of IgA antibody in either the saliva or in nasopharyngeal samples of persons exposed to the virus will be predictive of this recent exposure. Furthermore, the continuing presence of this antibody relates to the presence of the virus. Testing for the presence of the virus by a molecular assay will correlate with the specific IgA activity directed against the spike protein, RBD and nucleoprotein, as well as with identified viral peptides.

The rapid point of care (POC) assay of the invention is a lateral flow assay (LFA) where the saliva or nasopharyngeal sample is reacted with an antihuman IgA1 or IgA2 that has been labeled with colloidal gold and bound to an absorbent pad located on the test membrane. The sample migrates to a point on the membrane that has been striped with various viral components (RBD, Spike, S1, S2 and nucleocapsid) as well as selected viral peptides. An occurrence of a visible line indicates that there was IgA1 or IgA2 antibody present in the test sample that was targeted against a viral antigen (RBD, Spike, S1, S2, nucleocapsid) or viral peptides.

The rapid POC assay provides critical answers as to the initial exposure of an individual to the COVID-19 virus and his potential for being an asymptomatic carrier, as well as identifying the presence of targeted mucosal IgA in the saliva or nasopharynx sample of vaccinated individuals. In this application, the decrease in the amount of mucosal IgA will respond to an increasing potential for the asymptomatic carriage of the virus. It is the local targeted mucosal IgA1 and IgA2 response that inhibits viral spread both aerobically and systemically, and its reduction over time increases the likelihood of the vaccinated individual to become an asymptomatic carrier.

In summary, a test for the presence of IgA antibody directed against the Spike protein of the SARS-CoV-2 will be indicative of the presence of this virus although the carrier may be asymptomatic. The rapid POC assay (test) of the invention is easy to perform with results obtained within 10 minutes.

### SUMMARY OF THE INVENTION

As specified in the Background Section, there is a great need in the art to identify technologies for rapid POC assays for IgA antibodies directed against SARS-CoV-2 and use this understanding to develop novel diagnostic devices to specifically detect these specific IgA antibodies in subjects suspected of having COVID-19 or of asymptomatically carrying SARS-CoV-2. The present invention satisfies this and other needs. Embodiments of the invention relate generally to virus-specific assays and more specifically to lateral flow assays capable of detecting IgA antibodies specific for a component of SARS-CoV-2, such as for example, the Spike protein and/or RBD.

In one object of the invention there is provided a method for the detection of a mucosal IgAl and IgA2 antibody directed against a component of SARS-CoV-2 comprising the steps of i) contacting a biological sample with a colloidal gold labeled anti IgAl and IgA2 antibody to produce a reacted sample and ii) contacting the reacted sample with a SARS-CoV-2 viral component selected from the group consisting of the Spike protein, RBD, S1, S2 and viral peptides.

In a second object of the invention there is provided a method for the detection of an IgA antibody directed against the spike protein of SARS-CoV-2 virus (COVID-19 virus) or its components comprising the steps of i) contacting a biological sample with a colloidal gold labeled anti-human IgA1 and/or IgA2 antibody to produce a reacted sample and ii) contacting the reacted sample with a ‘capture’ line consisting of SARS-CoV-2 components selected from the group consisting of the spike, RBD, NP, S1, S2 and viral peptides.

In a third object of the invention a method for the detection of an asymptomatic carrier of SARS-CoV-2 virus comprising the steps of i) collecting a biological sample from a patient, and ii) contacting the biological sample with a colloidal gold anti-human IgA1 and IgA2 antibody to produce a reacted sample that is captured by a viral component selected from the group consisting of the spike, RBD, S1, S2, NP or viral peptides that has been striped on the lateral flow membrane.

In another object of the invention the biological sample is collected from a mucosal membrane.

In another object of the invention the biological sample is selected from the group consisting of saliva, mucus, sputum, phlegm, nasopharyngeal secretions, blood, serum, plasma, and urine. In an embodiment, the biological sample is selected from the group consisting of a nasopharyngeal swab, a throat swab, saliva, mucus, nasopharyngeal secretions and/or sputum.

In another object of the invention the biological sample is collected from a nasopharyngeal or a throat swab.

In a fourth object of the invention there is provided a lateral flow device for the detection of a human IgA antibody directed against the spike protein of SARS-CoV-2 virus (COVID-19 virus) or its truncated components comprising Spike protein or a truncated portion of the Spike protein wherein the receptor binding domain is used as a ‘capture’ line on the lateral flow membrane to entrap mucosal IgA that has been reacted with a gold tagged anti-human IgA1 and IgA2 antibody.

In another object of the invention there is provided a lateral flow device for the detection of a human mucosal IgA antibody directed against selected viral peptides. The viral peptides comprise a capture line that entraps the test sample that has been reacted with colloidal gold anti-human IgA1 and IgA2 antibody.

In another object of the invention there is provided a method for detecting an asymptomatic carrier state in a subject comprising determining the presence of mucosal IgA1 and IgA2 directed against a component of SARS-CoV-2 in biological samples.

In another object of the invention there is provided a method for measuring the presence of mucosal IgA in a biological sample from a subject who has received a vaccine against SAPS-CoV-2. Without wishing to be bound by theory, this assay may assist in determining the effect that decreasing amount of salivary IgA has on the vaccinated individual and his ability to still carry the virus in the nasopharynx asymptomatically.

In another object of the invention the lateral flow device further comprises a first capture line comprising an antibody specific for human IgA.

In another object of the invention the lateral flow device further comprises a second capture line comprising an antibody against a gold labeled antigen that is independent of any of the other reactants on the membrane.

These and other objects, features and advantages of the present invention will become more apparent upon reading the following specification in conjunction with the accompanying description, claims and drawings.

### BRIEF DESCRIPTION OF THE DRAWING

The FIGURE shows an exemplary lateral flow assay device according to the invention.

### DETAILED DESCRIPTION

As specified in the Background Section, there is a great need in the art to identify technologies for rapid POC assays for IgA antibodies directed against SARS-CoV-2 and use this understanding to develop novel diagnostic devices to specifically detect these specific IgA antibodies in subjects suspected of having COVID-19 or of asymptomatically carrying SARS-CoV-2. The present invention satisfies this and other needs. Embodiments of the invention relate generally to virus-specific assays and more specifically to lateral flow assays capable of detecting IgA antibodies specific for a component of SARS-CoV-2, such as for example, the Spike protein and/or RBD.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

An epidemic of respiratory disease caused by severe acute respiratory syndrome coronavirus 2 (SARS-CoV-2) has emerged as a major pandemic across the globe. Rapid and early SARS-CoV-2 diagnosis is essential for patient management and early disease intervention. A rapid test for the detection of IgA formed in response to SARS-CoV-2 in patients' biological samples is described in this patent.

Early and rapid diagnostic tools, management and confinement of SARS-CoV-2 cases are critical to halt virus transmission. A number of Reverse transcription-polymerase chain reaction (RT-PCR) tests to detect specific viral ribosomal nucleic acid (RNA) have recently been developed to detect SARS-CoV-2. These techniques unfortunately have a long turnaround time which can result in delayed diagnoses. They are also costly and require trained personnel to perform. Thus, their use is for large scale screening is limited.

The host humoral response against SARS-CoV-2 includes IgA, IgM and IgG. The median duration of IgM and IgA antibody detection were 5 days. The mucosal immune system and its predominant effector, secretory immunoglobulin A (IgA), provide the initial immunologic barriers against most pathogens that invade the body at a mucosal surface. This is especially true for viruses, since resistance to infection has been strongly correlated with the presence of specific IgA antibodies in mucosal secretions. Traditionally, the neutralization of viruses by immunoglobulins is thought to result from the binding of antibodies to virion attachment proteins, thereby preventing adherence to epithelial cells. In addition, mucosal antibodies interact intracellularly with viruses, preventing their replication, possibly by interfering with virus assembly.

The use of anti-Spike protein immunoglobulin A (IgA) as a diagnostic marker in saliva or mucosal surfaces that has not previously been explored. Recognizing the potential role of IgA in SARS-CoV-2 diagnosis, has allowed the development of a rapid new test that is capable of measuring mucosal IgA1 and IgA2 for the presence of SARS-CoV-2.

Pre-existing secretory IgA (S-IgA) antibodies can provide immediate immunity via their unique capability to eliminate a pathogen before it even passes the mucosal barrier and enters the human body, IgA has also been shown to be very effective at disarming viruses in virus- infected secretory epithelial cells and in redirecting antigens to the lumen when they enter the lamina propria. These responses are all non-inflammatory, since IgA, unlike IgG, does not fix complement and thus does not activate the inflammatory complement pathway. Therefore, a strong IgA response could be particularly important in case of highly pathogenic strains, where most complications are caused by uncontrolled pro-inflammatory responses.

### Devices of the Invention

In one aspect, the present invention provides a lateral flow assay (LFA) device capable of detecting the presence of IgA antibodies against SARS-CoV-2 in a subject who is suspected of having COVID-19, whether symptomatic or asymptomatic. In one embodiment, the device comprises antibodies that are specific for human IgA (e.g., IgA1 and IgA2) in combination with proteins unique to SARS-CoV-2, such as for example, Spike protein, RBD, NP, S1, and/or S2 peptides, including fragments, variations, and truncations thereof. In another embodiment, the device further comprises antibodies that are specific for human IgA, including human IgA1 and/or IgA2. In another embodiment, the device further comprises antibodies that are specific for a gold labeled antigen that is independent of any of the other reactants on the membrane.

The device comprises an absorbent membrane with proteins and/or antibodies bound to different regions of the membrane. In some embodiments, anti-human IgA antibodies are bound in a first region of the membrane, i.e., are closest to the end of the membrane where the biological sample is applied. The anti-human IgA antibodies can be labeled with any detector label known in the art, including but not limited to gold, chromogenic labels, and/or fluorescent labels. In some embodiments, one or more of SARS-CoV-2 Spike protein, RBD, NP, S1, and/or S2 peptides, including fragments, variations, and truncations thereof, are bound in a first or second region of the membrane, i.e., the second region is downstream of the first region. The SARS-CoV-2 proteins can be labeled with any detector label known in the art, including but not limited to gold, chromogenic labels, and/or fluorescent labels. In some embodiments, an antibody against a gold labeled antigen that is independent of any of the other reactants on the membrane is bound to another region of the membrane.

In some embodiments, the device is adapted for use with tagged antibodies to human IgA, meaning that the device comprises either (i) a single antibody region that is specific for human IgA; (ii) a region that comprises SARS-CoV-2 proteins, e.g., Spike protein, RBD, NP, S1, and/or S2 peptides, including fragments, variations, and truncations thereof; or (iii) both (i) and (ii). In some embodiments, the biological sample is mixed with a tagged or labeled anti-human IgA antibody (e.g., specific for IgA1 and/or IgA2) under conditions allowing the anti-human IgA antibody to bind to any IgA present in the sample. The antibody-antigen complex can then be introduced to the device comprising labeled SARS-CoV-2 proteins, e.g., Spike protein, RBD, NP, S1, and/or S2 peptides, including fragments, variations, and truncations thereof under conditions for the SARS-CoV-2 proteins to “capture” any IgA that is specific for SARS-CoV-2 virus in the sample.

In another embodiment, the device is adapted for use with colloidal gold particles that are labeled with antibodies specific to IgA (e.g., IgA1 and/or IgA2), meaning that the device comprises either i) a single antibody region that is specific for human IgA; (ii) a region that comprises SARS-CoV-2 proteins, e.g., Spike protein, RBD, NP, S1, and/or S2 peptides, including fragments, variations, and truncations thereof; or (iii) both (i) and (ii). In some embodiments, the biological sample is mixed with colloidal gold particles labeled with anti-human IgA antibody (e.g., specific for IgAl and/or IgA2) under conditions allowing the anti-human IgA antibody to bind to any IgA present in the sample. The antibody-antigen complex can then be introduced to the device comprising labeled SARS-CoV-2 proteins, e.g., Spike protein, RBD, NP, S1, and/or S2 peptides, including fragments, variations, and truncations thereof under conditions for the SARS-CoV-2 proteins to “capture” any IgA that is specific for SARS-CoV-2 virus in the sample.

In another embodiment, a device as described herein comprises a first region of the membrane comprising bound, tagged or labeled anti-human IgA antibodies in combination with a second region of the membrane comprising bound fragments, truncations and/or variations of the SARS-CoV-2 Spike protein, RBD, NP, S1, and/or S2 proteins. These SARS-CoV-2 proteins, including fragments, truncations and/or variations, are labeled or tagged such that interactions with the bound proteins result in a visual indication of the interaction, e.g., a chromogenic indication.

In another embodiment, a device as described herein comprises a first region of the membrane comprising bound, tagged or labeled anti-human IgA antibodies in combination with a second region of the membrane comprising bound SARS-CoV-2 viral particles. These viral particles are labeled or tagged such that interactions with the bound proteins result in a visual indication of the interaction, e.g., a chromogenic indication.

### Methods of Using the Devices

Generally, a specific gold labeled anti-human IgA1 and IgA2 antibody is dried on a menrmbrane. Of particular interest is the use of SARS-CoV-2 viral peptides that have been detected using linear peptide mapping of the entire proteome as a component of the capture line for entrapping the test sample with the gold labeled antibody. These SARS-CoV-2 peptides have been selected because they have extremely high activity against mucosal IgA1 and IgA2 from COVID infected patients. The use of the targeted viral peptides can change upon viral mutations. These SARS-CoV-2 variants can cause a change in the peptide array and can ensure the best test to detect mucosal IgA. A biological sample, such as for example, a nasopharyngeal swab, a throat swab, a saliva sample, a blood or blood product sample, a urine sample, a phlegm sample, a nasopharyngeal secretion sample, or a sputum sample, is added to a lateral flow device as described herein and makes initial contact with a labeled or tagged anti-human IgA antibody that has been immobilized on the membrane. The sample migrates until it makes contact with a line of labeled or tagged SARS-CoV-2 proteins, such as for example, Spike protein, RBD, NP, S1, and/or S2 peptides, including fragments, variations, and truncations thereof. Alternatively, the membrane can comprise labeled or tagged SARS-CoV-2 viral particles. A positive test, based on interaction with the labeled SARS-CoV-2 proteins or viral particles, develops a colored line or provides another visual indicator. In some embodiments, the membrane further comprises an antibody that is specific for a gold labeled antigen that is independent of any of the other reactants on the membrane. This independent antigen can provide a positive control for the assay.

In other embodiments, the invention includes the labeling of colloidal gold with other SARS-CoV-2 viral components, as well as tagging anti-human IgA antibodies with colloidal gold and utilizing the Spike protein, RBD (or other viral component) as the component of the capturing line.

In other embodiments, the invention includes detecting human IgA that is specific for one or more SARS-CoV-2 proteins, including truncations, fragments, and variants thereof. For example, the specific SARS-CoV-2 protein used in the lateral flow assay device can be the Spike protein and/or RBD, including fragments, truncations, and variants thereof. In such embodiments, the capture line of the lateral flow assay device comprises the specific SARS-CoV-2 protein(s), which is labeled or tagged.

In other embodiments, the biological sample is mixed with labeled or tagged antibodies that are specific for human IgA (including IgA1 and/or IgA2). The mixing is done under conditions suitable for the antibodies to bind to the target antigen. Once the incubation is complete, the biological sample is introduced to a lateral flow device as described herein, where the sample makes initial contact with a labeled or tagged protein and/or antibody that has been immobilized on the membrane. In some embodiments, the labeled or tagged protein is a SARS-CoV-2 protein, such as for example, Spike protein, RBD, NP, S1, and/or S2 peptides, including fragments, variations, and truncations thereof. Alternatively, the membrane can comprise labeled or tagged SARS-CoV-2 viral particles. A positive test, based on interaction with the labeled SARS-CoV-2 proteins or viral particles, develops a colored line or provides another visual indicator. In some embodiments, the membrane further comprises an antibody that is specific for a gold labeled antigen that is independent of any of the other reactants on the membrane. This independent antigen can provide a positive control for the assay.

In other embodiments, the biological sample is mixed with colloidal gold particles that are labeled with antibodies that are specific for human IgA. The mixing is done under conditions suitable for the particles to bind to the target antigen. Once the incubation is complete, the biological sample is introduced to a lateral flow device as described herein, where the sample makes initial contact with a labeled or tagged protein or antibody that has been immobilized on the membrane. If the colloidal gold particles used in the mixing step are labeled with anti-human IgA antibodies, then the immobilized proteins comprise SARS-CoV-2 proteins, such as for example, Spike protein, RBD, NP, S1, and/or S2 peptides, including fragments, variations, and truncations thereof. Alternatively, the membrane can comprise labeled or tagged SARS-CoV-2 viral particles. A positive test, based on interaction with the labeled SARS-CoV-2 proteins or viral particles, develops a colored line or provides another visual indicator. In some embodiments, the membrane further comprises an antibody that is specific for a gold labeled antigen that is independent of any of the other reactants on the membrane. This independent antigen can provide a positive control for the assay.

In other embodiments, the lateral flow device can comprise a first anti-human IgA antibody region in combination with a second region comprising either SARS-CoV-2 viral particles bound to the membrane, or fragments, truncations and/or variations of the SARS-CoV-2 Spike protein, RBD, NP, S1, and/or S2 proteins bound to the membrane. A biological sample is added to this lateral flow device comprising antibodies to IgA and SARS-CoV-2 viral particles or proteins. The biological sample makes initial contact with labeled or tagged anti-human IgA antibodies. The sample migrates until it makes contact with either the SARS-CoV-2 viral particles or with the SARS-CoV-2 viral proteins, including but not limited to Spike protein, RBD, NP, S1, and/or S2 peptides, as well as fragments, variations, and truncations thereof. A positive test, based on interaction with the labeled SARS-CoV-2 proteins or viral particles, develops a colored line or provides another visual indicator. In some embodiments, the membrane further comprises an antibody that is specific for a gold labeled antigen that is independent of any of the other reactants on the membrane. This independent antigen can provide a positive control for the assay.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Use of a Lateral Flow Device Comprising SARS-CoV-2 Viral Particles or Proteins

A biological sample is obtained from a subject suspected of having COVID-19, whether symptomatic or asymptomatic. The biological sample is a nasopharyngeal swab, a saliva sample, a blood or blood product sample, a urine sample, a phlegm sample, a nasopharyngeal secretion sample, or a sputum sample. The biological sample is added to a lateral flow device comprising SARS-CoV-2 viral particles or proteins, such that IgA specific for SARS-CoV-2 can be detected. The biological sample migrates until it makes contact with a line of labeled, tagged SARS-CoV-2 viral particles or labeled, tagged SARS-CoV-2 proteins, such as for example, Spike protein, RBD, NP, S1, and/or S2 peptides, including fragments, variations, and truncations thereof. A positive test, based on interaction with the labeled SARS-CoV-2 proteins or viral particles, develops a colored line or provides another visual indicator. In some embodiments, the membrane further comprises an antibody that is specific for a gold labeled antigen that is independent of any of the other reactants on the membrane. This independent antigen can provide a positive control for the assay.

### Example 2. Use of a Device Comprising SARS-CoV-2 Viral Particles or Proteins in Combination with Tagged Anti-Human IgA/IgA Complexes

A biological sample is obtained from a subject suspected of having COVID-19, whether symptomatic or asymptomatic. The biological sample is a nasopharyngeal swab, a saliva sample, a blood or blood product sample, a urine sample, a phlegm sample, a nasopharyngeal secretion sample, or a sputum sample. The biological sample is mixed with tagged or labeled. antibodies to human IgA (e.g., IgA1 and/or IgA2) under conditions allowing the antibody to bind with the desired antigen. After a suitable incubation, the sample is added to a lateral flow device comprising labeled or tagged SARS-CoV-2 viral particles or labeled or tagged SARS-CoV-2 proteins, such as for example, Spike protein, RBD, NP, S1, and/or S2 peptides, including fragments, variations, and truncations thereof. A positive test, based on interaction with the labeled SARS-CoV-2 proteins or viral particles, develops a colored line or provides another visual indicator. In some embodiments, the membrane further comprises an antibody that is specific for a gold labeled antigen that is independent of any of the other reactants on the membrane. This independent antigen can provide a positive control for the assay.

### Example 3. Use of a Device Comprising SARS-CoV-2 Viral Particles or Proteins in Combination with Colloidal Gold Particles Labeled with IgA

A biological sample is obtained from a subject suspected of having COVID-19, whether symptomatic or asymptomatic. The biological sample is a nasopharyngeal swab, a saliva sample, a blood or blood product sample, a urine sample, a phlegm sample, a nasopharyngeal secretion sample, or a sputum sample. The biological sample is mixed with colloidal gold particles comprising antibodies to human IgA (e.g., IgA1 and/or IgA2) under conditions allowing the antibody to bind with the desired antigen. After a suitable incubation, the sample is added to a lateral flow device comprising labeled or tagged SARS-CoV-2 viral particles or labeled or tagged SARS-CoV-2 proteins, such as for example, Spike protein, RBD, NP, S1, and/or S2. peptides, including fragments, variations, and truncations thereof. A positive test, based on interaction with the labeled SARS-CoV-2 proteins or viral particles, develops a colored line or provides another visual indicator. In some embodiments, the membrane further comprises an antibody that is specific for a gold labeled antigen that is independent of any of the other reactants on the membrane. This independent antigen can provide a positive control for the assay.

### Example 4. Use of a Device Comprising Antibodies to IgA and/or SARS-CoV-2 in Combination with Colloidal Gold Particles Labeled with SARS-CoV-2 Viral Particles or Proteins

A biological sample is obtained from a subject suspected of having COVID-19, whether symptomatic or asymptomatic. The biological sample is a nasopharyngeal swab, a saliva sample, a blood or blood product sample, a urine sample, a phlegm sample, a nasopharyngeal secretion sample, or a sputum sample. The biological sample is mixed with colloidal gold particles comprising SARS-CoV-2 viral particles or SARS-CoV-2 proteins, such as for example, Spike protein, RBD, NP, S1, and/or S2 peptides, including fragments, variations, and truncations thereof under conditions allowing the antibody to bind with the desired antigen. After a suitable incubation, the sample is added to a lateral flow device comprising labeled or tagged antibodies to either or both of human IgA and/or SARS-CoV-2 proteins, such as for example, Spike protein, RBD, NP, S1, and/or S2 peptides, including fragments, variations, and truncations thereof. A positive test, based on interaction with the labeled or tagged antibodies. In some embodiments, the membrane further comprises an antibody that is specific for a gold labeled antigen that is independent of any of the other reactants on the membrane. This independent antigen can provide a positive control for the assay.

### Example 5. Use of a Device Comprising Antibodies to IgA in Combination with SARS-CoV-2 Viral Particles or Proteins

A biological sample is obtained from a subject suspected of having COVID-19, whether symptomatic or asymptomatic. The biological sample is a nasopharyngeal swab, a saliva sample, a blood or blood product sample, a urine sample, a phlegm sample, a nasopharyngeal secretion sample, or a sputum sample. The sample is added to a lateral flow device comprising antibodies to IgA bound to a first region of the membrane. The lateral flow device comprises either SARS-CoV-2 viral particles or SARS-CoV-2 proteins, e.g., Spike protein, RBD, NP, S1, and/or S2 peptides, as well as fragments, variations, and truncations thereof. The biological sample makes initial contact with labeled or tagged anti-human IgA antibodies. The sample migrates until it makes contact with a line of labeled or tagged SARS-CoV-2 viral particles or proteins, such as for example, Spike protein, RBD, NP, S1, and/or S2 peptides, including fragments, variations, and truncations thereof. A positive test develops a colored line or provides another visual indicator. In some embodiments, the membrane further comprises an antibody that is specific for a gold labeled antigen that is independent of any of the other reactants on the membrane. This independent antigen can provide a positive control for the assay.

### Example 7. Use of a Device Comprising SARS-CoV-2 Viral Particles or Proteins to Detect and/or Quantify SARS-CoV-2 Specific IgA

A biological sample is obtained from a subject suspected of having COVID-19, whether symptomatic or asymptomatic. Alternatively, the biological sample can be obtained from a subject who has been vaccinated for SARS-CoV-2. The biological sample is a nasopharyngeal swab, a saliva sample, a blood or blood product sample, a urine sample, a phlegm sample, a nasopharyngeal secretion sample, or a sputum sample. The biological sample is added to a lateral flow device comprising SARS-CoV-2 viral particles or proteins, such that IgA specific for SARS-CoV-2 can be detected and quantified. In some embodiments, before the addition to the lateral flow device, the biological sample can be mixed with labeled or tagged (e.g., labeled with colloidal gold particles) anti-human IgA antibodies under conditions allowing the antibody to bind with the desired antigen. The biological sample migrates until it makes contact with a line of labeled, tagged SARS-CoV-2 viral particles or labeled, tagged SARS-CoV-2 proteins, such as for example, Spike protein, RBD, NP, S1, and/or S2 peptides, including fragments, variations, and truncations thereof. A positive test, based on interaction with the labeled SARS-CoV-2 proteins or viral particles, develops a colored line or provides another visual indicator. In some embodiments, the device enables the quantification of the amount of the IgA that is specific for SARS-CoV-2. In some embodiments, the membrane further comprises a labeled or tagged anti-human IgA antibody. In some embodiments, the membrane further comprises an antibody that is specific for a gold labeled antigen that is independent of any of the other reactants on the membrane. This independent antigen can provide a positive control for the assay.

### Example 8. Use of a Device Comprising a Certain SARS-CoV-2 Protein to Detect and/or Quantify IgA Specific for the Protein

A biological sample is obtained from a subject suspected of having COVID-19, whether symptomatic or asymptomatic. The biological sample is a nasopharyngeal swab, a saliva sample, a blood or blood product sample, a urine sample, a phlegm sample, a nasopharyngeal secretion sample, or a sputum sample. The biological sample is added to a lateral flow device comprising a specific SARS-CoV-2 protein, such that IgA specific for that protein can be detected and/or quantified. For example, the specific SARS-CoV-2 protein can be the Spike protein or RBD, including fragments, truncations, and variants thereof. In some embodiments, if the protein to be detected is the Spike protein, then the capture line can comprise tagged or labeled RBD. In some embodiments, before the addition to the lateral flow device, the biological sample can be mixed with labeled or tagged (e.g., labeled with colloidal gold particles) anti-human IgA antibodies under conditions allowing the antibody to bind with the desired antigen. The biological sample migrates until it makes contact with a line of labeled, tagged SARS-CoV-2 proteins, such as for example, Spike protein, RBD, NP, S1, and/or S2 peptides, including fragments, variations, and truncations thereof. A positive test, based on interaction with the labeled SARS-CoV-2 proteins, develops a colored line or provides another visual indicator. In some embodiments, the device enables the quantification of the amount of the IgA that is specific for the desired SARS-CoV-2 protein. In some embodiments, the membrane further comprises a labeled or tagged anti-human IgA antibody. In some embodiments, the membrane further comprises an antibody that is specific for a gold labeled antigen that is independent of any of the other reactants on the membrane. This independent antigen can provide a positive control for the assay.

While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims.

1. A method for the detection of an IgA antibody directed against a component of SARS-CoV-2 virus (COVID-19 virus) selected from the group consisting of Spike protein, RBD, NP, S1, and/or S2, and fragments, truncations, and variants thereof, comprising the steps of:
i) contacting a biological sample with a colloidal gold labeled anti-human IgA1 and IgA2 antibody to produce a reacted sample; and
ii) contacting the reacted sample with a lateral flow device comprising a SARS-CoV-2 virus capture line comprising one or more viral components selected from the group consisting of Spike protein, RBD, NP, S1, and/or S2, and fragments, truncations, and variants thereof.

2-4. (canceled)

5. The method according to claim 1, wherein the biological sample is collected from a mucosal membrane.

6. The method according to claim 1, wherein the biological sample is selected from the group consisting of saliva, mucus, phlegm, nasopharyngeal secretions, and sputum.

7. The method according to claim 1, wherein the biological sample is collected from a nasopharyngeal swab or a throat swab.

8. The method according to claim 1, wherein the lateral flow device further comprises a second capture line comprising an antibody against a gold labeled antigen that is independent of any of the other reactants on the membrane.

9. The method according to claim 1, wherein the lateral flow device further comprises a region comprising an anti-human IgA antibody.

10-14. (canceled)

14. A lateral flow assay device for the detection of human IgA antibody directed against SARS-CoV-2 viral particles or proteins, including fragments, truncations, or variants of the viral proteins, the lateral flow device comprising:
an anti-human IgA1 and IgA2 tagged with colloidal gold dried on a pad which is affixed to a membrane of the lateral flow device; and
a capture line on the membrane comprising SARS-CoV-2 viral particles or proteins, including fragments, truncations, or variants of the viral proteins,
wherein the SARS-CoV-2 viral proteins are selected from the group consisting of Spike protein, RBD, NP, S1, and/or S2.

15. The lateral flow assay device according to claim 14 further comprising a first capture line comprising an antibody specific for detecting human mucosal IgA antibody that is reactive with SARS-CoV-2 viral particles or proteins, including fragments, truncations, or variants of the viral proteins, wherein the SARS-CoV-2 viral proteins are selected from the group consisting of Spike protein, RBD, NP, S1, and/or S2.

16. The lateral flow assay device according to claim 14 further comprising a second capture line comprising an antibody against a gold labeled antigen that is independent of any of the other reactants on the membrane.

17. The lateral flow assay device according to claim 14 further comprising a region comprising an anti-human IgA antibody.

18. (canceled)

19. A method for identifying a subject as having or being an asymptomatic carrier of SARS-CoV-2 comprising:
i) contacting a biological sample from the subject with a colloidal gold labeled anti-human IgA antibody to produce a reacted sample;
ii) contacting the reacted sample with a lateral flow device comprising a capture line comprised of SARS-CoV-2 viral particles or SARS-CoV-2 Spike protein, RBD, NP, S1, and/or S2, and fragments, truncations, and variants thereof; and
determining the presence of IgA specific for SARS-CoV-2 based on a visual indication of the reacted sample binding to the capture line.

20. The method according to claim 19, wherein the biological sample is collected from a mucosal membrane.

21. The method according to claim 19, wherein the biological sample is selected from the group consisting of saliva, mucus, phlegm, nasopharyngeal secretions, and sputum.

22. The method according to claim 19, wherein the biological sample is collected from a nasopharyngeal swab or a throat swab.

23. The method according to claim 19, wherein the lateral flow device further comprises a second capture line comprising an antibody against a gold labeled antigen that is independent of any of the other reactants on the membrane.

24. The method according to claim 19, wherein the lateral flow device further comprises a region comprising an anti-human IgA antibody.

25-30. (canceled)
